(19) United States
(12) Patent Application Publication
(10) Pub. No.: US 2014/0283214 A1
(43) Pub. Date: Sep. 18, 2014

(54) MAIZE SUGARY ENHANCER SEQUENCES

(71) Applicant: Wisconsin Alumni Research Foundation, Madison, WI (US)

(72) Inventors: Shawn M. Kaeppler, Oregon, WI (US); Candice Nicole Hirsch, Saint Paul, MN (US); Karl Joseph Haro von Mogel, Madison, WI (US)

(73) Assignee: Wisconsin Alumni Research Foundation, Madison, WI (US)

(21) Appl. No.: 14/216,613

(22) Filed: Mar. 17, 2014

Related U.S. Application Data
(60) Provisional application No. 61/791,200, filed on Mar. 15, 2013.

(51) Int. Cl. C12N 15/82; C12Q 1/68

(52) U.S. Cl. CPC C12N 15/8245; C12Q 1/6895
USPC 800/284; 435/6.11; 800/320.1; 435/412

(57) ABSTRACT

The sugary enhancer phenotype in maize results from the absence of expression of a nucleotide sequence located on Chromosome 2. Methods for screening, selecting or identifying plants containing the sequence and for estimating the sucrose content in maize plants are disclosed. Genetically modified plants having altered expression of the nucleotide sequence are provided.

### CROSS REFERENCE TO RELATED APPLICATIONS

This application claims the benefit of U.S. Provisional Application No. 61/791,200, filed Mar. 15, 2013, herein incorporated by reference in its entirety.

### INCORPORATION OF SEQUENCE LISTINGS

The sequence listing contained in the file named “WARF108US_ST25.txt”, which is 18 KB (as measured in Microsoft Windows®) and was created on Mar. 17, 2014, is filed herewith by electronic submission and is incorporated by reference herein.

### BACKGROUND

Sugary Enhancer (se1) is a gene involved in starch metabolism in maize endosperms and is found in varieties grown on approximately half of the sweet corn acreage in the United States. It is a modifier of the Sugary (su1) gene, but its mode of action is unknown. The Sugary1 (Su1) locus encodes an isoamylase-type starch debranching enzyme (DBE) which removes excess branches from growing amylopectin molecules, allowing the formation of an efficiently-packed starch granule. Recessive su1 alleles increase the sucrose content of the endosperm at the expense of amylopectin, and the loss of Su1 debranching activity increases the number of branches in the growing starch molecules, leading to the production of the water soluble polysaccharide (WSP) known as phytoglycogen. WSP in maize endosperm produces a desirable creamy texture in sweet corn at its eating stage. The sucrose content of sweet corn can be further increased with the addition of other recessive alleles of starch pathway genes, such as shrunken-2 (sh2), but this may reduce the levels of WSP and thus alter the texture.

When se1 is combined with su1, it increases the levels of sucrose and other simple sugars in the endosperm, while not significantly reducing the levels of WSP characteristic of Sugary endosperm. The high levels of water-soluble polysaccharides in Sugary and Sugar Enhanced sweet corn provides a creamy texture to the kernel. Sugar Enhanced sweet corn has sugar levels similar to Supersweet (sh2) corn. “se type” sweet corn also has tender kernels, lighter color, and good flavor. se-type sweet corn varieties trace to a line called IL677a, developed in the 1970s from a cross between two sweet corn lines (IL44b, IL442a) and a Carioco flour corn population (Bolivia 1035).

Expression intensity of the se1 trait is dependent on genetic background which has prevented its mapping using routine techniques. Previous studies have genetically mapped se1 to different locations and chromosomes in the maize genome. This disparity concerning the location of se1 is attributed to the difficulty of scoring the presence of se1 in dry seeds, which has slowed the development of high-quality se1 type sweet corns.

### SUMMARY

In some embodiments, methods of estimating the sucrose content in a maize plant having kernels are provided. An assay is performed to detect the presence or absence of a polynucleotide encoding a polypeptide having at least 90% identity to SEQ ID NO: 4 or at least about 90% identity to SEQ ID NO: 5 in a sample comprising the plant, or part thereof. The presence of the sequence correlates with decreased sucrose in the kernels and the absence of the sequence correlates with increased sucrose in the kernels and enhanced palatability. The assay may include a polymerase chain reaction in which the sample is contacted by a polymerase and at least two different nucleotide primers for the amplification of at least a portion of the polynucleotide. The assay may include contacting the sample with a labeled nucleotide that binds to the polynucleotide and detecting the presence or absence of nucleotide. In some embodiments, detecting the presence or absence of the polynucleotide comprises the detection of a deletion of the polynucleotide sequence from a region of maize genomic DNA within 1.8 Mb of SSR marker UMC 1736. In some embodiments detecting the presence or absence of the polynucleotide comprises the detection of a deletion of the polynucleotide sequence from a region of maize genomic DNA between genetic markers Agt1 and UMC 1736. In other embodiments, detection of the deletion of the polynucleotide comprises detecting the presence of a genetic marker genetically linked to the deletion.

In some embodiments, methods of identifying or selecting a maize plant cell having increased sucrose are provided. The presence or absence of a molecular marker in the cell is detected in a cell that has been contacted with the molecular marker. The molecular marker is associated with the presence or absence of a polynucleotide encoding a polypeptide having at least 90% identity with SEQ ID NO: 4 or with SEQ ID NO: 5. The presence of polynucleotide correlates with decreased sucrose in the kernels and the absence the polynucleotide correlates with increased sucrose in the kernels. The maize plant cell can have the su1 genotype.

In some embodiments, methods for altering the amount of sucrose in a maize kernel of a maize plant are provided. The methods include altering expression of a polypeptide which is encoded by a polynucleotide. The polypeptide has at least 90% identity with SEQ ID NO: 4 or SEQ ID NO: 5. The expression of the polypeptide and thus the amount of sucrose in the kernel is altered by introducing a mutation into the polynucleotide, wherein the mutation reduces or prevents expression of the polypeptide, or by introducing a nucleic acid molecule into the maize plant, wherein the nucleic acid molecule reduces or prevents expression of the polypeptide. The maize plant can have the su1 genotype.

In other embodiments, a genetically modified maize plant cell or maize plant having the su1 genotype is provided. The plant cell or plant contains a foreign nucleic acid that decreases expression of a polypeptide having at least 90% homology to SEQ ID NO: 4 or SEQ ID NO: 5. The plant or plant cell has increased sucrose compared with a corresponding maize plant or plant cell having the su1 genotype that does not comprise the foreign nucleic acid. The foreign nucleic acid can include a nucleic acid molecule encoding at least one antisense RNA that reduces the expression of at least one endogenous gene encoding the polypeptide, a nucleic acid molecule which, via a co-suppression effect, reduces the expression of at least one endogenous gene encoding the polypeptide, or a nucleic acid molecule that simultaneously encodes at least one antisense RNA and at least one sense RNA, where said antisense RNA and said sense RNA form a double-stranded RNA molecule that reduces the expression of the polypeptide.

In some embodiments, a method of selecting a maize plant having kernels is provided. The method includes detecting the presence or absence of a bound polynucleotide that binds to a genomic nucleotide sequence corresponding to position 910 to 1514 of SEQ ID NO: 1 or to position 910 to 1524 of SEQ ID NO: 2 in a sample comprising the plant, or part thereof, which has been contacted with the polynucleotide. The binding of the polynucleotide to the genomic nucleotide sequence correlates with decreased sucrose in the kernels and an absence of binding of the polynucleotide to the genomic nucleotide sequence correlates with increased sucrose in the kernels.

Other aspects of the invention will become apparent by consideration of the detailed description and accompanying drawings.

### BRIEF DESCRIPTION OF THE DRAWINGS

FIG. 1 is a photograph showing the phenotype in the mapping population. a. Segregating ear showing smooth Se1 (homozygous and heterozygous) and wrinkled se1 (homozygous) dry seeds. Note the lighter color of the wrinkled seeds, which is often associated with se1. b. Se1 seeds show an opaque phenotype when viewed on a lightbox, while se1 seeds show a translucent phenotype.

FIG. 2 is a diagram showing the genetic mapping of se1. a. Location of Se1 region on chromosome 2 indicated by red box near the distal end. b. First zoom level of Se1 region in B73 showing 1.26 Mb between the markers AGT1 and UMC 1736. Mapping markers and the number of recombinant plants with the Se1 genotype from the mapping population are indicated by the vertical lines and associated numbers. c. Second zoom level showing 24 kb surrounding Se1, with gene models indicated by the blue and yellow bars. d. Mapping data from 3 proximal and 3 distal recombinants (identified at left). Vertical lines indicate marker locations. The red bar represents regions heterozygous for Se1 and se1 genotypes, light blue indicates se1 homozygous genotype, and purple indicates unknown genotype between markers. The genotypes of recombinants at the Se1 locus were determined by sequencing. All recombinants shown and homozygous se1/se1 controls possessed the deletion indicated by the gray box and dotted lines, and all other gene models were excluded by the markers used for fine mapping.

FIG. 3 is a photograph showing the semi-quantitative RT-PCR of Se1 compared to an expression standard in several tissues and developmental time points in B73. Expression was observed in developing leaves (V5 base of stage-2 leaf), whole kernels from 12-24 DAP, and endosperm (12 DAP). Histone acetyltransferase complex component (GRMZM2G129817) was used as an expression standard for the same samples. The larger 250 base pair band in the control gDNA lane includes a small intron, whose absence from the other lanes indicates negligible DNA contamination in these samples.

FIG. 4 is a graph showing mature leaf starch data from a 24-hour time course in the field, between Se1 and se1 genotypes. Means are indicated by symbols, and bars indicate one standard deviation from the mean. Time is in 24 hour notation, with sunrise at 5:30 and sunset at 20:30.

FIG. 5 is a diagram showing a comparison of the sugary enhancer region in lines B73, Se and se.

FIG. 6 is a diagram showing the alignment of the nucleotide coding sequences for the Se1 polypeptide in lines B73 and Se1 along with upstream and downstream sequences in the B73 genome. The corresponding region from the se1 line which lacks the coding sequence is also shown. Exemplary primer positions are shown underlined, and the start and stop codons are highlighted in bold.

FIG. 7 is a diagram showing the alignment of the Se1 polypeptide in lines B73 and Se1.

### DETAILED DESCRIPTION

A nucleotide sequence in the maize genome has been identified as encoding a polypeptide that confers the sugary enhancer (se1) phenotype in maize plants. The genomic sequence comprises 519 bases (or 522 bases including the stop codon) in the maize line B73 and 552 bases (or 555 bases including the stop codon) in the maize line Se1. In plants having the se1 phenotype, the se1 allele is deleted. The genomic sequence does not include introns. Plants or plant cells expressing a polypeptide encoded by the nucleotide sequence have reduced sucrose compared with plants that do not express the polypeptide, particularly when the recessive sugary (su1) allele is present such that the plants or cells do not express the isoamylase-type starch debranching enzyme (DBE) encoded at the Su1 locus. Plants or plant cells which do not express or show reduced expression of the polypeptide have increased sucrose compared with plants that do not express the polypeptide, particularly when the recessive sugary (su1) allele is present, such that the plants or cells do not express the isoamylase-type starch debranching enzyme (DBE) encoded at the Su1 locus.

The plants that can be used with the methods, compositions and kits disclosed herein are typically maize (Zea mays) plants. However, other monocotyledonous plants such as rice, wheat, rye, barley, oats, sugar cane, sorghum, or grass may be used if sequence comparable to Se1 is present in varieties of these species. Plants include whole plants and plant parts, including plant cells, kernels, kernel cells, leaves, stems, roots, grains, tubers, fruits, flowers and inflorescence.

Methods, kits and compositions comprising molecular markers which facilitate identification of the presence or absence of the Se1 or B73 allele are provided. The molecular marker can be any molecule which facilitates detection of the presence or absence of the Se1 coding region. For example, the molecular marker may include primer sequences that facilitate the identification of the presence or absence of the Se1 coding region in a polymerase chain reaction (PCR). The assay can be performed by contacting the sample with a polymerase and at least two, at least three, at least four, at least five or at least six different nucleotide primers for the amplification of the polynucleotide or a portion of the polynucleotide. In certain embodiments, at least one, at least two, or at least three, at least four, at least five or at least six of the primers binds to a portion of one or more of SEQ ID NOs: 1-3.

In certain embodiments, a primer sequence is provided which anneals or binds to a region substantially upstream of the Se1 gene and can be used for the forward (left) primer; a primer sequence is selected which anneals or binds to a region substantially downstream of the deleted region of se1 (corresponding to position 910 to position 1514 of SEQ ID NO: 1) to be used for the reverse (right) primer; and/or a primer sequence is selected which anneals or binds to a region which is at least substantially or wholly within the deleted region in se1 to be used as the second reverse primer. The reverse primer which detects Se1 preferably lies wholly or substantially in the deleted region of se1 (corresponding to position 910 to position 1514 of SEQ ID NO: 1; position 910 to position 1524 of SEQ ID NO: 2, or combination thereof) such that it does not amplify in se1 lines. Preferably, the reverse primer which detects Se1 lies outside of the high-GC content in the middle of the Se1 gene sequence, enabling regular Taq polymerase to amplify the region. If a Taq polymerase is used that is able to amplify the high GC content in the middle of the Se1 gene, the primer from anywhere substantially inside of the deleted region may be used. The reverse primer which detects Se1 can also be used as a forward (left) primer to pair up with another second reverse (right) primer. Exemplary primers are shown in SEQ ID NO: 6-8 and are annotated in FIG. 6. The reverse complement of any primer sequence described herein can also be used.

Primer SEQ ID NO: 6 anneals in the Se1 and B73 genotypes, and in other functional alleles. SEQ ID NO: 8 anneals in B73, Se1 and se1. However, the Se1 gene has a relatively high G/C content of approximately 80%, such that many Taq polymerases used in PCR will not amplify across the Se1 gene. Accordingly, while SEQ ID NO: 8 or an equivalent primer from the proximal region anneals in both Se1 and se1, it will produce a PCR product in the se1 genotype but not in the Se1 genotype. Primer combinations can be used such that when the PCR reaction is run out on a gel, one size band is obtained for the presence of Se1, a different, for example larger, band is obtained for se1, and two bands are obtained if the DNA sample comes from a plant that is heterozygous and has one of each allele. Thus, primers can be designed using the present disclosure to produce a co-dominant marker which shows when both versions of the allele are present.

The molecular markers described and used herein, may include molecules, such as nucleic acid molecules or nucleotide sequences, which hybridize or otherwise bind to the polynucleotide encoding Se1, or to regions in proximity to the sequence, such as those disclosed in SEQ ID NOs: 1-3. The molecular marker may include a tag or label, such as fluorescent (or fluorogenic), luminescent (or luminogenic) or radioactive tag or label.

The molecular markers may be selected to hybridize or bind to a region that is up to about 100 nucleotides upstream or downstream of the Se1 gene, up to about 200 nucleotides upstream or downstream of the Se1 gene, up to about 300 nucleotides upstream or downstream of the Se1 gene, up to about 400 nucleotides upstream or downstream of the Se1 gene, up to about 500 nucleotides upstream or downstream of the Se1 gene, up to about 600 nucleotides upstream or downstream of the Se1 gene, up to about 700 nucleotides upstream or downstream of the Se1 gene, up to about 800 nucleotides upstream or downstream of the Se1 gene, up to about 900 nucleotides upstream or downstream of the Se1 gene, up to about 1,000 nucleotides upstream or downstream of the Se1 gene, up to about 1,250 nucleotides upstream or downstream of the Se1 gene, up to about 1,500 nucleotides upstream or downstream of the Se1 gene, up to about 1,750 nucleotides upstream or downstream of the Se1 gene, up to about 2,000 nucleotides upstream or downstream of the Se1 gene, up to about 2,500 nucleotides upstream or downstream of the Se1 gene, up to about 3,000 nucleotides upstream or downstream of the Se1 gene, up to about 3,500 nucleotides upstream or downstream of the Se1 gene, up to about 4,000 nucleotides upstream or downstream of the Se1 gene, up to about 4,500 nucleotides upstream or downstream of the Se1 gene, up to about 5,000 nucleotides upstream or downstream of the Se1 gene, up to about 6,000 nucleotides upstream or downstream of the Se1 gene, up to about 7,000 nucleotides upstream or downstream of the Se1 gene, up to about 8,000 nucleotides upstream or downstream of the Se1 gene, up to about 9,000 nucleotides upstream or downstream of the Se1 gene, up to about 10,000 nucleotides upstream or downstream of the Se1 gene, up to about 12,500 nucleotides upstream or downstream of the Se1 gene, up to about 15,000 nucleotides upstream or downstream of the Se1 gene, up to about 17,500 nucleotides upstream or downstream of the Se1 gene, up to about up to about 20,000 nucleotides upstream or downstream of the Se1 gene, nucleotides upstream or downstream of the Se1 gene, up to about 25,000 nucleotides upstream or downstream of the Se1 gene, up to about 30,000 nucleotides upstream or downstream of the Se1 gene, up to about 40,000 nucleotides upstream or downstream of the Se1 gene, up to about 50,000 nucleotides upstream or downstream of the Se1 gene or up to about 100,000 nucleotides upstream or downstream of the Se1 gene.

In certain embodiments, methods of estimating the sucrose content in plants, plant cells, kernels or plant kernel cells are provided. An assay can be performed to detect the presence or absence of a polynucleotide encoding a polypeptide disclosed herein, such as a polypeptide having at least 90% identity to SEQ ID NO: 4 or to SEQ ID NO: 5. The presence of the sequence correlates with decreased sucrose in the kernels and the absence of the sequence correlates with increased sucrose in the kernels. The assay may include a polymerase chain reaction in which the sample is contacted a polymerase and at least two different nucleotide primers for the amplification of at least a portion of the polynucleotide. The assay may include contacting the sample with a labeled nucleotide that binds to the polynucleotide and detecting the presence or absence of nucleotide.

The term “substantial identity” of amino acid sequences (and of polypeptides having these amino acid sequences) normally means sequence identity of at least 40% compared to a reference sequence as determined using the programs described herein; preferably BLAST using standard parameters, as described herein. Preferred percent identity of amino acids can be any integer from 40% to 100%. More preferred embodiments include amino acid sequences that have at least about: 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference sequence. Polypeptides that are “substantially identical” share amino acid sequences as noted above except that residue positions which are not identical may differ by conservative amino acid changes. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, aspartic acid-glutamic acid, and asparagine-glutamine. Accordingly, polypeptides or proteins of the present invention include amino acid sequences that have substantial identity to the amino acid sequences of the polypeptides of the present invention, which are modified phytochromes that result in plants having altered sensitivity compared with plants.

Two nucleic acid sequences or polypeptides are said to be “identical” if the sequence of nucleotides or amino acid residues, respectively, in the two sequences are the same when aligned for maximum correspondence as described below. The term “complementary to” is used herein to mean that the sequence is complementary to all or a portion of a reference polynucleotide sequence. In the case of both expression of transgenes and inhibition of endogenous genes (e.g., by antisense or sense suppression) the inserted polynucleotide sequence need not be identical and may be “substantially identical” to a sequence of the gene from which it was derived.

In the case of polynucleotides used to inhibit expression of an endogenous gene, the introduced sequence need not be perfectly identical to a sequence of the target endogenous gene. The introduced polynucleotide sequence will typically be at least substantially identical (as determined below) to the target endogenous sequence.

In the case where the inserted polynucleotide sequence is transcribed and translated to produce a functional polypeptide, because of codon degeneracy, a number of polynucleotide sequences will encode the same polypeptide. These variants are specifically covered by the term “polynucleotide sequence from” a particular gene. In addition, the term specifically includes sequences (e.g., full length sequences) that are substantially identical (determined as described below) with a gene sequence encoding a polypeptide of the present invention and that encode polypeptides or functional polypeptide fragments that retain the function of a polypeptide of the present invention, e.g., a modified bacterial phytochrome with increased fluorescence.

Optimal alignment of sequences for comparison may be conducted by methods commonly known in the art, for example by the search for similarity method described by Pearson and Lipman 1988, Proc. Natl. Acad. Sci. USA 85: 2444-2448, by computerized implementations of algorithms such as GAP, BESTFIT, BLAST, FASTA, and TFASTA in the Wisconsin Genetics Software Package, Genetics Computer Group (GCG), Madison, Wis., or by inspection. In a preferred embodiment, protein and nucleic acid sequence identities are evaluated using the Basic Local Alignment Search Tool (“BLAST”), which is well known in the art (Karlin and Altschul, 1990, Proc. Natl. Acad. Sci. USA 87: 2267-2268; Altschul et al., 1997, Nucl. Acids Res. 25: 3389-3402), the disclosures of which are incorporated by reference in their entireties. The BLAST programs identify homologous sequences by identifying similar segments, which are referred to herein as “high-scoring segment pairs,” between a query amino or nucleic acid sequence and a test sequence which is preferably obtained from a protein or nucleic acid sequence database. Preferably, the statistical significance of a high-scoring segment pair is evaluated using the statistical significance formula (Karlin and Altschul, 1990). The BLAST programs can be used with the default parameters or with modified parameters provided by the user.

“Percentage of sequence identity” is determined by comparing two optimally aligned sequences over a comparison window, where the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 25% sequence identity compared to a reference sequence as determined using the programs described herein; preferably BLAST using standard parameters, as described. Alternatively, percent identity can be any integer from 25% to 100%. More preferred embodiments include polynucleotide sequences that have at least about: 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98% or 99% sequence identity compared to a reference sequence. These values can be appropriately adjusted to determine corresponding identity of proteins encoded by two nucleotide sequences by taking into account codon degeneracy, amino acid similarity, reading frame positioning, and the like. Accordingly, polynucleotides of the present invention encoding a protein of the present invention include nucleic acid sequences that have substantial identity to the nucleic acid sequences that encode the polypeptides of the present invention. Polynucleotides encoding a polypeptide comprising an amino acid sequence that has at least about: 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, or 99% sequence identity compared to a reference polypeptide sequence are also preferred.

In some embodiments, methods of identifying or selecting a plant cell, such as a maize plant cell having the su1 genotype, and having increased sucrose are provided. The presence or absence of a molecular marker is detected in the cell that has been contacted with the molecular marker. The molecular marker is associated with the presence or absence of a polynucleotide encoding a polypeptide sharing a percentage identity, as described herein, with SEQ ID NO: 4 or with SEQ ID NO: 5. When the polynucleotide is present in the plant the molecular marker facilitates its detection. The presence of polynucleotide correlates with decreased sucrose in the kernels and the absence the polynucleotide correlates with increased sucrose in the kernels. In certain embodiments, the presence or absence of the marker associated with the polynucleotide can be detected in subsequent generations of the plant facilitating selection of the trait over multiple generations.

The absence of the se1 polynucleotide may increase sucrose, or other simple sugars such as glucose, fructose or maltose, in the plant, plant cell, kernel or plant cell kernel, such as one having the su1 genotype, by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 90%, at least about 95% or at least about 100%.

In some embodiments, a method for altering the amount of sucrose in a maize kernel of a maize plant, such as a maize plant comprising the su1 genotype, is provided. The maize plant contains an endogenous Se1 polynucleotide, such as SEQ ID NO: 8 or SEQ ID NO: 9, such as encoding SEQ ID NO: 4 or SEQ ID NO: 5 or a polypeptide sharing a percentage identity, as described herein, with SEQ ID NO: 4 or with SEQ ID NO: 5. The expression of the polypeptide is altered by introducing a mutation into the polynucleotide which reduces or prevents expression of the polypeptide. Alternatively, a nucleic acid molecule is introduced into the maize plant that reduces or prevents expression of the polypeptide and increases the amount of sucrose in the plant.

Mutations introduced into the polynucleotide to disrupt expression of the polypeptide encoded by the polynucleotide include, without limitation, one or more point mutations, deletions or insertions. Nucleic acid molecules introduced into the maize plant to reduce expression of the polypeptide include nucleotides which cause antisense suppression, sense suppression, or a combination thereof.

Genetically modified maize plants and plant cells are provided which express a foreign nucleic acid that decreases expression of SEQ ID NO: 4 or with SEQ ID NO: 5 or with a polypeptide sharing a percentage identity, as described herein, with SEQ ID NO: 4 or with SEQ ID NO: 5. The maize plant or plant cell can include the su1 recessive genotype and has increased sucrose compared with a corresponding maize plant or plant cell having the su1 genotype that does not comprise the foreign nucleic acid.

The foreign nucleic acid that decreases expression of the polypeptide can include a nucleic acid molecule encoding at least one antisense RNA that reduces the expression of at least one endogenous gene encoding the polypeptide; a nucleic acid molecule which, via a co-suppression effect, reduces the expression of at least one endogenous gene encoding the polypeptide; or a nucleic acid molecule that simultaneously encodes at least one antisense RNA and at least one sense RNA, where said antisense RNA and said sense RNA form a double-stranded RNA molecule that reduces the expression of the polypeptide.

It will be apparent to those of skill in the art that variations may be applied to the compositions and methods described herein and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

It is to be understood that the invention is not limited in its application to the details of construction and the arrangement of components set forth in the following description. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The entire disclosure of each document cited (including patents, patent applications, patent publications, journal articles, abstracts, laboratory manuals, books, or other disclosures) as well as information available through Identifiers specific to databases such as GenBank, GeneSeq, or the CAS Registry, referred to in this application are herein incorporated by reference in their entirety.

The following examples are for purely illustrative purposes and are not to be construed as limiting the scope of the invention.

### EXAMPLE 1

### Materials and Methods

Genetic Stocks:

Near-isogenic lines segregating for se1 were developed from an initial cross between Wh8419 and Terminator, a commercial hybrid. The F1 progeny were self-pollinated, and the resulting F2 were self-pollinated for 13 additional generations. FIG. 1: shows a segregating ear with both smooth Se1 (homozygous and heterozygous) and wrinkled se1 (homozygous) dry seeds (a). The wrinkled seeds had a lighter color than the smooth seeds. The opaque and translucent characteristics of Se1 and se1 kernels respectively were apparent when viewed on a light box. FIG. 1 b. These materials which formed the mapping population were maintained as heterozygotes and selected for low sugar content.

Carbohydrate Analysis:

Two side-by-side plots of homozygous Se and se plants from the mapping population were grown in the summer of 2011, from which both leaf and ear samples were taken. Leaf tissue samples were taken from the leaf below the flag leaf, which were 10 inches in length, starting at 2 inches from the base of the leaf. Leaves were photographed and the samples were chopped into 1-inch lengths, frozen in liquid nitrogen, and transferred to −80° C. Three biological replications were collected for each genotype at each sample time point. Leaf samples were collected for a 24-hour time course every 3 hours, beginning at dawn (0530) on July 8, and ending at dawn on July 9. Samples were also collected at 1130 on several subsequent dates (July 11, 13, 14, 15, 17, 18 19). Leaf samples for carbohydrate analysis were chopped and lyophilized, and ground into powder to obtain 100 mg of dry tissue.

Se and se ears were self-pollinated and reciprocal crosses were made. Three random ears were harvested and frozen at −20° C. for each genotype and reciprocal cross, at 2-day intervals between 14 and 24 days after pollination (DAP). Whole seeds from mature ears (45 DAP) of Se1 and se1 genotypes that were grown side-by-side in 2006 and 2007 were also analyzed with three biological replications each.

The kernels were freeze-dried and ground using an Udy mill with a 0.5 mm screen. The ground seed tissue was used to determine individual sugars and polysaccharides.

Sucrose, D-fructose, and D-glucose were measured using the Megazyme Sucrose, D-Fructose, and D-Glucose assay kit (commercially available from Megazyme International Ireland, Ltd., Bray, Ireland). 100 mg of ground seed sample was placed in a 15 mL glass tube, which was suspended in water and centrifuged. The supernatant was removed and used for sugar analysis. The supernatant was adjusted to 1.0 mg of seed sample per 1.0 mL of water, and 0.1 mL of this solution was used for analysis.

Sucrose was determined by adding β-fructosidase, NADP+ (150 mg) plus ATP, imidazole buffer and reading the absorption of the solution at 340 nm. Hexokinase (425 U/mL) plus glucose-6-phosphate dehydrogenase (212 U/mL) suspension was then added and the solution was again read at 340 nm. Blank and sucrose standards were also included in the assay.

D-fructose and D-glucose was determined by adding NADP+ (150 mg) plus ATP, imidazole buffer and reading the absorbance at 340 nm. hexokinase (425 U/mL) plus glucose-6-phosphate dehydrogenase (212 U/mL) suspension and phosphoglucose isomerase suspension was added and the absorbance was measured after each addition. A D-fructose/D-glucose and a blank control were also included in the assay. Concentrations of blank samples and controls were used to convert changes in absorbance to mg g-1 of dry weight of sucrose, D-fructose, and D-glucose.

Total polysaccharides and starch were measured using the Megazyme K-TSTA assay kit (catalog number K-TSTA, Megazyme International Ireland Ltd., Bray, Ireland). For both assays, 100 mg of ground seed sample was placed in a 15 mL glass tube. Two individual washes of the ground seed tissue were done using 80% v/v aqueous ethanol. After ethanol addition, the tubes were centrifuged and the ethanol was poured off. For the starch assay, 5 additional washes of 10% v/v aqueous ethanol followed. Following the ethanol washes, thermostable α-amylase and amyloglucosidase was added. This enzymatic step was used for the total polysaccharide and starch assay to hydrolyze the glucose linkages and break the samples down to glucose monomers.

After the enzymatic reaction, the solution was adjusted to 100 mL with water. Aliquots of this solution were mixed with glucose oxidase/peroxidase (GOPOD) and the absorbance was measured at 510 nm. Maize starch was included as a control for the enzymatic step while a glucose control and water sample were included for the glucose measurement. The absorbance was measured for each sample and converted to mg g-1 of dry weight.

Genetic Analysis:

DNA Oligonucleotide primers were designed using genomic DNA from the B73 reference sequence, obtained from maizesequence.org. Genes and gene fragments were identified based on evidence of expression from Expressed Sequence Tags (ESTs), and/or by gene models in open reading frames predicted by FGENESH. Primers were designed using Primer3 to flank introns. Homozygous smooth (Se/Se) and homozygous wrinkled (se/se) seeds from the mapping population were germinated from three different ears for each genotype, and DNA was extracted with a CTAB protocol. These DNA samples were used as three biological replications (bio-reps) for both Set and se1 genotypes. Primer pairs were amplified by Polymerase Chain Reaction (PCR) with a touch-down protocol using HotStarTaq. High GC-content sequences were amplified using a touch-down protocol using AccuPrime. Successful amplifications were Sanger sequenced, and the data was analyzed using BioEdit. Polymorphisms were screened as markers against the mapping population of se/se seeds using a variety of methods including RFLP, direct sequencing, and KASPar SNP assays. Tables 1a and 1b summarize these markers.

TABLE 1a
Genotypes of recombinant plants grown from wrinkled seeds on selfed segregating Se/se heterozygous ears. Blank cells reflect
genotypes that could not be determined after multiple attempts. Genotypes begin as heterozygous Se/se
on proximal marker AGT1, and progress to homozygous se/se at se deletion marker.

| Coordinates | | | | | | | | |
|---|---|---|---|---|---|---|---|---|
| 229608585 | 229850328 | 229936451 | 229946578 | 229949601 | 229966248 | 229975454 | 229977331 | 229981917 |

| | Marker Type | | | | | | | | |
|---|---|---|---|---|---|---|---|---|---|
| | RFLP | SNP | SNP | GBS | GBS | RFLP | SNP | Indel | GBS |
| | Marker Name | | | | | | | | |
| | | ME | ME | | ME | MF | MG | MG | MF |
| Individual | AGT1 | 11 | 61 | ME 4 | 68 | 20 | 12 | 15 | 29 |
| Se/Se | Se/Se | Se/Se | Se/Se | Se/Se | Se/Se | Se/Se | Se/Se | Se/Se | Se/Se |
| control | | | | | | | | | |
| se/se | se/se | se/se | se/se | se/se | se/se | se/se | se/se | se/se | se/se |
| control | | | | | | | | | |
| C 41 | Se/se | Se/se | Se/se | se/se | se/se | | Se/se | se/se | se/se |
| C 125 | Se/se | Se/se | se/se | se/se | | se/se | se/se | se/se | se/se |
| C 151 | Se/se | Se/se | se/se | se/se | se/se | se/se | se/se | se/se | se/se |
| C 152 | Se/se | Se/se | se/se | | se/se | se/se | se/se | se/se | se/se |
| C 159 | Se/se | se/se | se/se | se/se | | se/se | se/se | se/se | se/se |
| C 160 | Se/se | se/se | se/se | se/se | se/se | se/se | se/se | se/se | se/se |
| C 213 | Se/se | | se/se | se/se | se/se | se/se | se/se | se/se | se/se |
| 22 | Se/se | | se/se | se/se | se/se | se/se | se/se | | |
| 36 | Se/se | | | se/se | se/se | se/se | | | se/se |
| 49 | Se/se | | | se/se | se/se | se/se | | | |
| 60 | Se/se | se/se | se/se | se/se | se/se | se/se | se/se | se/se | se/se |
| 62 | Se/se | Se/se | se/se | se/se | se/se | se/se | se/se | se/se | se/se |
| 129 | Se/se | | Se/se | | Se/se | Se/se | Se/se | Se/Se | Se/se |
| 184 | Se/Se | Se/Se | se/se | se/se | se/se | se/se | se/se | se/se | se/se |
| 210 | Se/se | se/se | se/se | se/se | se/se | se/se | se/se | se/se | se/se |
| 250 | Se/se | Se/se | se/se | se/se | se/se | se/se | se/se | se/se | se/se |
| 257 | Se/se | Se/se | Se/se | se/se | se/se | se/se | Se/se | se/se | se/se |
| 297 | Se/se | se/se | se/se | se/se | Se/se | Se/se | se/se | Se/Se | Se/se |
| 313 | Se/se | se/se | se/se | se/se | se/se | se/se | se/se | se/se | se/se |
| 372 | Se/se | | se/se | se/se | se/se | se/se | se/se | se/se | se/se |
| 506 | Se/se | se/se | se/se | se/se | se/se | se/se | se/se | se/se | se/se |
| 522 | Se/se | se/se | se/se | se/se | se/se | se/se | se/se | se/se | se/se |
| 394 | Se/Se | se/se | se/se | se/se | | se/se | se/se | se/se | se/se |
| 399 | Se/Se | se/se | se/se | se/se | se/se | se/se | se/se | se/se | se/se |
| 400 | Se/Se | | se/se | | | se/se | se/se | | se/se |
| C 197 | Se/se | | Se/se | se/se | se/se | se/se | Se/se | Se/Se | se/se |
| C 209 | Se/se | | Se/se | se/se | se/se | se/se | Se/se | Se/Se | se/se |
| 549 | Se/Se | Se/se | se/se | se/se | se/se | se/se | se/se | se/se | se/se |
| 550 | Se/Se | Se/se | se/se | se/se | se/se | se/se | se/se | se/se | se/se |
| 569 | Se/Se | | se/se | se/se | se/se | se/se | se/se | se/se | se/se |
| 576 | Se/Se | | Se/se | Se/se | Se/se | Se/se | | Se/Se | se/se |
| | Coordinates | | | | | | | | |
| | 229982958 | 229983835 | 229984097 | 229985046 | 229989916 | 230000900 | 230524884 | 230917249 | 230873138 |
| | Marker Type | | | | | | | | |
| | Deletion | RFLP | SNP | Indel | GBS | RFLP | SNP | RFLP | SSR |
| | Marker Name | | | | | | | | |
| | MF36F30R | MF | MF | MG77 | ME | MD | | MA | UMC |
| Individual | DEL2R | 3933 | 39 | 57 | 37 | 27 | MC 4 | 15 | 1736 |
| Se/Se | Se/Se | Se/Se | Se/Se | Se/Se | Se/se | Se/Se | Se/Se | Se/Se | Se/Se |
| control | | | | | | | | | |
| se/se | se/se | se/se | se/se | se/se | se/se | se/se | se/se | se/se | se/se |
| control | | | | | | | | | |
| C 41 | se/se | se/se | se/se | se/se | se/se | se/se | se/se | se/se | se/se |
| C 125 | se/se | | se/se | | se/se | | se/se | se/se | se/se |
| C 151 | se/se | se/se | se/se | se/se | se/se | se/se | se/se | se/se | se/se |
| C 152 | se/se | se/se | se/se | se/se | se/se | se/se | se/se | | se/se |
| C 159 | se/se | se/se | se/se | se/se | se/se | se/se | se/se | se/se | se/se |
| C 160 | se/se | se/se | se/se | se/se | se/se | se/se | se/se | | se/se |
| C 213 | se/se | se/se | se/se | se/se | se/se | se/se | se/se | | se/se |
| 22 | se/se | se/se | | se/se | se/se | se/se | se/se | se/se | se/se |
| 36 | | se/se | | se/se | se/se | | | se/se | se/se |
| 49 | | se/se | Se/Se | se/se | se/se | se/se | | se/se | se/se |
| 60 | se/se | se/se | se/se | se/se | se/se | se/se | se/se | se/se | se/se |
| 62 | se/se | se/se | se/se | se/se | se/se | se/se | se/se | se/se | se/se |
| 129 | se/se | se/se | se/se | | se/se | se/se | se/se | se/se | se/se |
| 184 | se/se | se/se | se/se | se/se | se/se | se/se | se/se | se/se | se/se |
| 210 | se/se | se/se | se/se | se/se | se/se | | se/se | se/se | se/se |
| 250 | se/se | se/se | se/se | se/se | se/se | se/se | se/se | se/se | se/se |
| 257 | se/se | se/se | se/se | se/se | se/se | se/se | se/se | se/se | se/se |
| 297 | se/se | se/se | se/se | se/se | se/se | se/se | se/se | se/se | se/se |
| 313 | se/se | se/se | se/se | se/se | se/se | se/se | se/se | se/se | se/se |
| 372 | se/se | se/se | se/se | se/se | se/se | se/se | se/se | se/se | se/se |
| 506 | se/se | se/se | se/se | se/se | se/se | se/se | se/se | se/se | se/se |
| 522 | se/se | se/se | se/se | se/se | se/se | se/se | Se/Se | Se/Se | Se/Se |
| 394 | se/se | se/se | se/se | se/se | se/se | se/se | se/se | se/se | se/se |
| 399 | se/se | se/se | se/se | se/se | se/se | se/se | se/se | se/se | se/se |
| 400 | se/se | se/se | se/se | se/se | se/se | se/se | se/se | se/se | se/se |
| C 197 | se/se | se/se | | se/se | se/se | se/se | Se/se | se/se | se/se |
| C 209 | se/se | se/se | | se/se | se/se | se/se | Se/se | se/se | se/se |
| 549 | se/se | se/se | se/se | se/se | se/se | | Se/Se | Se/Se | Se/Se |
| 550 | se/se | se/se | se/se | se/se | se/se | se/se | Se/Se | Se/Se | Se/Se |
| 569 | se/se | se/se | se/se | se/se | se/se | se/se | se/se | se/se | se/se |
| 576 | se/se | se/se | se/se | se/se | se/se | se/se | se/se | se/se | se/se |

TABLE 1b
Genotypes of recombinant plants grown from wrinkled seeds on selfed segregating Se/se heterozygous ears. Blank cells reflect
genotypes that could not be determined after multiple attempts. Genotypes begin as heterozygous Se/se on
distal marker UMC1736 and progress to homozygous se/se at se deletion marker.

| | Coordinates | | | | | | | | |
|---|---|---|---|---|---|---|---|---|---|
| | 229608585 | 229850328 | 229936451 | 229946578 | 229949601 | 229966248 | 229975454 | 229977331 | 229981917 |
| | Marker Type | | | | | | | | |
| | RFLP | SNP | SNP | GBS | GBS | RFLP | SNP | Indel | GBS |
| | Marker Name | | | | | | | | |
| | | ME | ME | | ME | MF | MG | MG | MF |
| Individual | AGT1 | 11 | 61 | ME 4 | 68 | 20 | 12 | 15 | 29 |
| Se/Se | Se/se | Se/Se | Se/Se | Se/Se | Se/Se | Se/Se | Se/Se | Se/Se | Se/Se |
| control | | | | | | | | | |
| se/se | se/se | se/se | se/se | se/se | se/se | se/se | se/se | se/se | se/se |
| control | | | | | | | | | |
| C 51 | se/se | se/se | se/se | se/se | se/se | se/se | se/se | se/se | se/se |
| C 106 | se/se | se/se | se/se | se/se | se/se | se/se | se/se | | se/se |
| C 173 | se/se | | se/se | se/se | se/se | | se/se | | se/se |
| C 201 | se/se | se/se | se/se | se/se | se/se | se/se | se/se | Se/Se | se/se |
| 2 | se/se | se/se | se/se | | se/se | se/se | se/se | | se/se |
| 10 | se/se | | se/se | | se/se | se/se | se/se | | se/se |
| 50 | se/se | | se/se | | se/se | se/se | se/se | se/se | |
| 79 | se/se | | se/se | se/se | se/se | se/se | se/se | se/se | se/se |
| 97 | se/se | se/se | se/se | se/se | se/se | se/se | se/se | se/se | se/se |
| 104 | se/se | | se/se | se/se | se/se | se/se | se/se | se/se | se/se |
| 107 | se/se | | se/se | | se/se | | | | |
| 110 | se/se | se/se | se/se | se/se | se/se | se/se | se/se | se/se | se/se |
| 202 | Se/Se | Se/Se | se/se | se/se | se/se | se/se | se/se | se/se | se/se |
| 213 | se/se | se/se | se/se | se/se | se/se | se/se | se/se | se/se | se/se |
| 225 | se/se | se/se | se/se | se/se | se/se | se/se | se/se | se/se | se/se |
| 248 | se/se | se/se | se/se | se/se | se/se | se/se | se/se | se/se | se/se |
| 292 | se/se | se/se | se/se | se/se | se/se | se/se | se/se | se/se | se/se |
| 304 | se/se | se/se | se/se | se/se | se/se | se/se | se/se | se/se | se/se |
| 316 | se/se | se/se | se/se | se/se | se/se | se/se | se/se | se/se | se/se |
| 318 | se/se | se/se | se/se | se/se | se/se | se/se | se/se | se/se | se/se |
| 381 | se/se | | se/se | | se/se | se/se | se/se | se/se | |
| 411 | se/se | se/se | se/se | se/se | se/se | se/se | se/se | se/se | se/se |
| 468 | se/se | se/se | | se/se | se/se | se/se | se/se | se/se | se/se |
| 469 | se/se | se/se | se/se | se/se | se/se | se/se | se/se | se/se | se/se |
| 472 | se/se | se/se | se/se | se/se | se/se | se/se | se/se | se/se | se/se |
| 508 | se/se | se/se | se/se | se/se | se/se | se/se | se/se | se/se | se/se |
| 509 | se/se | se/se | se/se | se/se | se/se | se/se | se/se | se/se | se/se |
| 514 | se/se | se/se | se/se | se/se | Se/se | se/se | se/se | se/se | |
| | Coordinates | | | | | | | | |
| | 229982958 | 229983835 | 229984097 | 229985046 | 229989916 | 230000900 | 230524884 | 230917249 | 230873138 |
| | Marker Type | | | | | | | | |
| | Deletion | RFLP | SNP | Indel | GBS | RFLP | SNP | RFLP | SSR |
| | Marker Name | | | | | | | | |
| | MF36F30R | MF | MF 39 | MG77 | ME | MD | | MA | UMC |
| Individual | DEL2R | 3933 | nt294 | 57 | 37 | 27 | MC 4 | 15 | 1736 |
| Se/Se | Se/Se | Se/Se | Se/Se | Se/Se | Se/Se | Se/Se | Se/Se | Se/Se | Se/Se |
| control | | | | | | | | | |
| se/se | se/se | se/se | se/se | se/se | se/se | se/se | se/se | se/se | se/se |
| control | | | | | | | | | |
| C 51 | se/se | se/se | se/se | se/se | se/se | se/se | se/se | Se/se | Se/se |
| C 106 | se/se | se/se | se/se | se/se | se/se | | se/se | Se/se | Se/se |
| C 173 | se/se | se/se | se/se | se/se | se/se | se/se | se/se | Se/se | Se/se |
| C 201 | se/se | se/se | se/se | Se/se | Se/se | Se/Se | Se/se | Se/se | Se/se |
| 2 | se/se | se/se | se/se | se/se | se/se | se/se | se/se | Se/se | Se/se |
| 10 | | se/se | se/se | se/se | se/se | se/se | se/se | Se/se | Se/se |
| 50 | | se/se | se/se | se/se | se/se | se/se | Se/se | Se/se | Se/Se |
| 79 | | se/se | se/se | se/se | se/se | | se/se | se/se | Se/se |
| 97 | se/se | se/se | se/se | se/se | se/se | se/se | Se/se | Se/se | Se/se |
| 104 | se/se | se/se | se/se | se/se | se/se | se/se | Se/se | Se/se | Se/se |
| 107 | se/se | se/se | se/se | se/se | se/se | se/se | Se/se | Se/se | Se/se |
| 110 | se/se | se/se | se/se | se/se | se/se | se/se | se/se | Se/se | Se/se |
| 202 | se/se | se/se | se/se | se/se | se/se | se/se | se/se | Se/se | Se/Se |
| 213 | se/se | se/se | se/se | se/se | se/se | se/se | Se/se | Se/se | Se/se |
| 225 | se/se | se/se | se/se | se/se | se/se | se/se | Se/se | Se/se | Se/se |
| 248 | se/se | se/se | se/se | se/se | se/se | se/se | Se/se | Se/se | Se/se |
| 292 | se/se | se/se | se/se | se/se | se/se | se/se | Se/se | Se/se | Se/se |
| 304 | se/se | se/se | se/se | se/se | se/se | se/se | Se/se | Se/se | Se/se |
| 316 | se/se | se/se | se/se | se/se | se/se | se/se | Se/se | Se/se | Se/se |
| 318 | se/se | se/se | se/se | se/se | se/se | se/se | se/se | Se/se | Se/se |
| 381 | se/se | se/se | se/se | se/se | se/se | se/se | Se/se | Se/se | Se/se |
| 411 | se/se | se/se | se/se | se/se | se/se | se/se | se/se | Se/se | Se/se |
| 468 | se/se | se/se | se/se | se/se | se/se | se/se | Se/se | Se/se | Se/se |
| 469 | se/se | se/se | se/se | se/se | se/se | se/se | se/se | Se/se | Se/se |
| 472 | se/se | se/se | se/se | se/se | se/se | se/se | se/se | Se/se | Se/se |
| 508 | se/se | se/se | se/se | se/se | Se/se | Se/Se | Se/se | Se/se | Se/se |
| 509 | se/se | se/se | se/se | se/se | se/se | se/se | se/se | Se/se | Se/se |
| 514 | se/se | se/se | se/se | se/se | | se/se | Se/Se | Se/Se | Se/se |

Expression Analysis:

RNA was extracted from B73 plants at specific stages of development using a Trizol extraction protocol. cDNA was synthesized using the SuperScript III kit according to the manufacturer's directions. GRMZM2G129817 was selected as a low-expression control from Sekhon et al., and primers were designed flanking a small intron to produces a small (size) band from cDNA, and a larger band that includes the intron from genomic DNA. The semi-quantitative PCR reaction was performed using AccuPrime with 20 cycles of touchdown followed by 22 cycles with a constant annealing temperature, which were determined through testing to be the optimal number of cycles to reveal differences in expression.

### EXAMPLE 2

### Identification of se Phenotype

A near-isogenic mapping population was developed that discriminates between homozygous recessive se seeds, which appear wrinkled, and dominant Se seeds, which appear smooth. Self-pollinated ears on heterozygous plants segregate in a 3:1 ratio, as expected from a simple recessive trait. Test crosses between these materials and a homozygous Se1 and a homozygous se1 plant yielded 100% smooth kernels, and 100% wrinkled kernels for each cross respectively. The mapping population is homozygous for sugaryl (su1).

An analysis of the carbohydrate content of mature seeds and developing kernels in the mapping population was performed, which revealed clear differences between the genotypes. Mature seeds (45 DAP) from three homozygous selfed Se ears grown in 2006 and 2007 averaged 42.3 and 40.5% starch, respectively, while homozygous se seeds from 3 ears grown in nearby rows on the same years averaged 22 and 22.6% starch. WSP in Se seeds averaged 13.7 and 16.1%, while se seeds were significantly higher at 26.8% for both years. Sucrose was 48% higher in se genotypes compared to Se, while total polysaccharides and total carbohydrates were slightly lower.

Whole, developing kernels were analyzed for carbohydrate content at the eating stage (22 DAP) in 3 ears each from self-pollinated Se and se plants. Reciprocal crosses between the two genotypes were also created to produce all four combinations of Se copy number in the endosperm. The results are shown in Table 2.

TABLE 2

| | | | | | | Total | | | Total | Total |
|---|---|---|---|---|---|---|---|---|---|---|
| Genotype | DAP | Year | Glucose | Fructose | Sucrose | Sugars | WSP | Starch | Poly. | Carb. |
| Se | 45 | 2006 | 1.27 | 1.26 | 3.43 | 5.96 | 13.70 | 42.31 | 56.01 | 61.97 |
| Se | 45 | 2007 | 1.47 | 1.17 | 3.71 | 6.35 | 16.08 | 40.53 | 56.61 | 62.96 |
| se | 45 | 2006 | 1.40 | 1.16 | 4.99 | 7.54 | 26.79 | 21.96 | 48.74 | 56.28 |
| se | 45 | 2007 | 1.36 | 1.31 | 5.60 | 8.28 | 26.77 | 22.60 | 49.37 | 57.65 |
| Se | 22 | 2011 | 4.67 | 4.84 | 6.25 | 15.76 | 15.40 | 35.01 | 50.40 | 66.16 |
| Se × se | 22 | 2011 | 4.91 | 5.06 | 5.72 | 15.70 | 11.96 | 38.03 | 49.99 | 65.69 |
| se × Se | 22 | 2011 | 4.18 | 4.09 | 5.52 | 13.80 | 19.62 | 34.74 | 54.36 | 68.16 |
| se | 22 | 2011 | 5.35 | 5.22 | 7.71 | 18.27 | 26.39 | 19.40 | 45.79 | 64.06 |

### EXAMPLE 3

### Mapping

Sugary Enhancer 1 (Se1) was mapped to the distal end of the long arm of Chromosome 2 by association with UMC 1736, an SSR marker. An additional marker was identified, Agt1, which placed Se1 between these two markers with Agt1 on the proximal side and UMC 1736 on the distal side, in a region that is 1.8 Mb in length. A screen of 820 homozygous se-type seeds (1640 gametes) from segregating ears identified 63 recombinants for one or both markers. Screening at 15 additional markers fine mapped Se1 to a narrow interval containing a single gene model, AC217415.3_FG004 (FIG. 2), with the nearest crossovers within 300 base pairs of the proximal and distal sides of the gene.

Se1 contains a 522 base pair open reading frame (ORF) in B73 that has a high (80%) GC content, and encodes for a predicted peptide that is 173 amino acid residues in length. There are no introns within the ORF, and based upon expression evidence from ESTs the gene consists of only one exon with no introns. One EST encompasses the Poly-A tail, which defines the 3′ end of the transcript. The encoded peptide is Glycine, Alanine, and Arginine-rich (36, 29, & 22 of 173 residues), and is 17.4 kD in mass. There are some known sequence variations at this locus in different inbred lines, including Single Nucleotide Polymorphisms (SNPs) and 3-6 base pair in-frame indels (small insertions and deletions). The sugary enhancer region in lines B73, Se and se is shown in more detail in FIG. 5.

The SE1 protein does not have a high incidence of Serine or Threonine residues in the N-terminus, which would be indicative of a Chloroplast or other plastid signaling peptide. TargetP rates the likelihood of Chloroplast targeting and export low, mitochondrial destination medium, and other destinations to be high. ChloroP rates its chloroplast signaling as medium-low (0.443), and Motif Scan results indicate a possible bipartite nuclear localization signal (RRVVFRAERDGGRLRLR) (SEQ ID NO: 11), consisting of three Arginine and two Lysine residues in an alternating fashion. However, there is only one part of a two-part signal so this does not appear to indicate that Se1 is localized to the nucleus.

A 24.3 kb region encompassing Se1 and the nearest proximal and distal gene models was sequenced in both Se1 and se1 genotypes. This sequence revealed a significant number of SNPs, as well as many small indels. A few major indels were observed, most notably a 637 base pair deletion in the se genotype which completely eliminates the ORF of Se1. Another notable polymorphism is a 647 base pair deletion that is approximately 1.3 kb upstream (proximal) from the beginning of the transcript, which is shared by both the Se and se genotypes when compared to B73. Third, an 836 base pair deletion is present in se approximately 2.3 kb on the distal side of the gene, and at the same location in Se1 there are 29 unique bases in the place of 189 bases present in B73. The region distal to Se1 is more highly polymorphic than the proximal side, however both contain a significant number of SNPs.

### EXAMPLE 4

### Analysis of the Homology of Se1

An analysis of the homology of Se1 was performed and revealed several similar genes in other grass species, but none were found outside the monocots. Sorghum bicolor possesses the closest nucleotide match, Sb05g025625.1, with 64% coverage and a maximum identity of 90%. LOC_Os11g42410.1 in rice (Oryza sativa japonica) and Si027819m.0 in foxtail millet (Setaria italica) have short amino acid sequence similarities of only 9-15 residues. None of these similar genes have any annotation information that indicates function.

Within the Zea mays L. genome there is one significant inverted alignment to GRMZM5G842214 on Chromosome 1, which with GRMZM2G159316 forms an overlapping pair of short genes that are antisense with respect to each other. GRMZM2G159316 also has a strong match to a region that is approximately 1 kb upstream of the transcription start site of Du1, a starch synthase III.

### EXAMPLE 5

### Expression Pattern of Se1

The tissue-specific expression pattern of Se1 was obtained with semi-quantitative RT-PCR performed on RNA from a range of B73 tissues (FIG. 3). Expression of Se1 was low in whole seeds at 4 DAP, but increased at 12 DAP to a maximum at 16-20 DAP, and appeared to level off at 24 DAP. A 12 DAP endosperm sample confirmed that it is endosperm-expressed, and a sample taken from the base of a Stage-2 V5 leaf showed relatively strong expression in developing adult leaf tissue. Expression was low in root, seedling, internode, silk, and stem/SAM. Histone acetyltransferase complex component, which was used as an expression standard, had constant low expression in all tissues.

Therefore Se1 was a low-expressed gene overall. This tissue-specific expression pattern in endosperm and developing leaves is confirmed by RNA-Seq data available for this transcript on the Maize Genome Database, placing the maximum observed expression of Se1 at 8-14 fragments per kilobase of exon per million fragments mapped (FPKM). Currently available RNA seq data also indicates expression in the developing embryo. The absence of a larger (250 base pair) band in the expression standard lanes indicates the absence of DNA contamination of the RNA samples.

### EXAMPLE 6

### Analysis of Leaf Carbohydrates

An analysis of leaf starch from the 24-hour time course demonstrated a diurnal pattern of sucrose, free sugar, and starch accumulation beginning at dawn, and a decline in leaf carbohydrates beginning at dusk. Only one significant difference was found in starch levels between Se1 and se1 genotypes at one time point (1430), as determined by a two-tailed T-test (p=0.044). The Se1 genotype had starch levels at 2.5% by dry weight, while se1 was slightly higher at 3.2%. No other significant differences were found between the two genotypes at the other time points, so this difference was not a consistent phenotype. (FIG. 4) Sucrose and other free sugars were not significantly different between the genotypes at any time point, and a comparison of carbohydrate levels in leaves sampled on subsequent days with varying weather patterns and temperatures found no significant differences between the genotypes.

Listing of Sequences

SEQ ID NO: 1 Polynucleotide from maize (Zea mays) line B73 showing the Se1 coding sequence starting at position 944 and ending at position 1462 prior to stop codon with upstream and downstream genomic sequences.

SEQ ID NO: 2 Polynucleotide from maize (Zea mays) line Se1 showing the Se1 coding sequence starting at position 944 and ending at position 1495 prior to stop codon with upstream and downstream genomic sequences.

SEQ ID NO: 3 Polynucleotide from maize (Zea mays) line se1 showing the genomic sequence surrounding the deleted Se1 coding sequence.

SEQ ID NO: 4 Se1 polypeptide from maize (Zea mays) line B73 encoded by positions 944 to 1462 of SEQ ID NO: 1 (SEQ ID NO: 9).

SEQ ID NO: 5 Se1 polypeptide encoded by SEQ ID NO: 1 encoded by positions 944 to 1495 of SEQ ID NO: 2. (SEQ ID NO: 10)

SEQ ID NO: 6 Nucleotide primer which binds to region shown underlined in FIG. 6 (1/3).

SEQ ID NO: 7 Nucleotide primer which binds to region shown underlined in FIG. 6 (2/3).

SEQ ID NO: 8 Nucleotide primer which binds to the region shown underlined in FIG. 6 (3/3).

SEQ ID NO: 9 Se1 coding sequence from line B73

SEQ ID NO: 10 Se1 coding sequence from line Se1

SEQ ID NO: 11 Possible bipartite nuclear localization signal in SE1

What is claimed is:

1. A method of estimating the sucrose content in a maize plant, the method comprising;
performing an assay to detect the presence or absence of a polynucleotide encoding a polypeptide having at least 90% identity to SEQ ID NO: 4 or at least about 90% identity to SEQ ID NO: 5 in a sample from the plant;
wherein detection of the sequence indicates low sucrose content in the maize plant, and wherein absence of the sequence indicates high levels of sucrose in the maize plant.

2. The method of claim 1, wherein performing the assay comprises contacting the sample with a polymerase and at least two different nucleotide primers for the amplification of the polynucleotide or a portion of the polynucleotide.

3. The method of claim 2, wherein at least one of the primers binds to a portion of one or more of SEQ ID NOs: 1-3.

4. The method of claim 3, wherein at least one of the primers binds to:
a) a sequence from position 1 to position of 909 SEQ ID NO:1, from position 910 to 1514 of SEQ ID NO: 1, from position 910 to 1514 of SEQ ID NO:1, from position 910 to 1524 of SEQ ID NO: 2, or combination thereof; or
b) the reverse complement of the sequence from position 1 to position of 909 SEQ ID NO:1, from position 910 to 1514 of SEQ ID NO: 1, from position 910 to 1514 of SEQ ID NO:1, from position 910 to 1524 of SEQ ID NO: 2, or combination thereof.

5. The method of claim 3, wherein the primers comprise:
a) SEQ ID NO: 6, SEQ ID NO: 7, SEQ ID NO: 8, or a combination thereof or,
b) a reverse complement of SEQ ID NO: 6, SEQ ID NO: 7, SEQ ID NO: 8, or a combination thereof.

6. The method of claim 1, wherein detecting the presence or absence of the polynucleotide comprises the detection of a genetic marker associated with the presence or absence of the polynucleotide.

7. The method of claim 1, wherein detecting the presence or absence of the polynucleotide comprises detecting the presence or absence of a deletion in the Se1 locus.

8. The method of claim 1, wherein performing the assay comprises contacting the sample with a labeled nucleotide that binds to the polynucleotide and detecting the presence or absence of nucleotide.

9. The method of claim 1, wherein the sample from the plant comprises a plant cell or a polynucleotide extract of a plant cell.

10. A method of identifying or selecting a maize plant cell having increased sucrose, the method comprising;
detecting the presence or absence of a molecular marker in the cell; and
identifying or selecting a maize plant cell when the molecular marker is absent;
wherein the molecular marker is associated with the presence of a polynucleotide encoding a polypeptide having at least 90% identity with:
(a) SEQ ID NO: 4; or
(b) SEQ ID NO: 5.

11. The method of claim 10, further comprising detecting the presence or absence of the molecular marker in one or more subsequent generations of the maize plant.

12. The method of claim 10, wherein the molecular marker comprises an insertion, a deletion, or a single nucleotide polymorphism.

13. A method for altering the amount of sucrose in a maize kernel of a maize plant comprising a polynucleotide encoding a polypeptide having at least 90% identity with (a) SEQ ID NO: 4 or (b) SEQ ID NO: 5, the method comprising altering the expression of the polypeptide by:
(a) introducing a mutation into the polynucleotide, wherein the mutation reduces or prevents expression of the polypeptide; or
(b) introducing a nucleic acid molecule into the maize plant, wherein the nucleic acid molecule reduces or prevents expression of the polypeptide;
thereby altering the amount of sucrose in the kernel.

14. The method of claim 13, wherein a mutation is introduced into the polynucleotide which is a point mutation, deletion or insertion, and wherein the amount of sucrose in the kernel is increased.

15. The method of claim 13, wherein the nucleic acid molecule is introduced into the maize plant and wherein the nucleic acid molecule reduces expression of the polypeptide by: (i) antisense suppression, (ii) sense suppression, or (iii) a combination thereof; thereby increasing the amount of sucrose in the kernel.

16. The method of claim 13, wherein the maize plant comprises the su1 genotype.

17. A genetically modified maize plant cell comprising a foreign nucleic acid that decreases expression of a polypeptide having at least 90% identity to (a) SEQ ID NO: 4 or (b) SEQ ID NO: 5, the maize plant cell having the su1 genotype and having increased sucrose compared with a corresponding maize plant cell having the su1 genotype that does not comprise the foreign nucleic acid.

18. The plant cell of claim 17, wherein the foreign nucleic acid that decreases expression of the polypeptide comprises (i) a nucleic acid molecule encoding at least one antisense RNA that reduces the expression of at least one endogenous gene encoding the polypeptide; (ii) a nucleic acid molecule which, via a co-suppression effect, reduces the expression of at least one endogenous gene encoding the polypeptide; or (iii) a nucleic acid molecule that simultaneously encodes at least one antisense RNA and at least one sense RNA, where said antisense RNA and said sense RNA form a double-stranded RNA molecule that reduces the expression of the polypeptide.

19. A maize plant comprising the genetically modified plant cell of claim 17.

20. A method of selecting a maize plant having kernels with increased sucrose, the method comprising:
detecting the presence or absence of a bound polynucleotide that binds to a genomic nucleotide sequence corresponding to position 910 to 1514 of SEQ ID NO: 1 or to position 910 to 1524 of SEQ ID NO: 2 in a sample from the plant; and
selecting the plant when the bound polynucleotide is absent.


SEQUENCE LISTING (11 sequences)

SEQ ID NO: 1 - DNA, 2392 bp, Zea mays
ctgaacctctttaaaattgtcagtgtgctagttccctattaggggatgacaattggtatgaaacttgtggtacacgggttagaaaccggtactcactaaacccgcactcacatctattaccccgtgacgagtacaatatatttaggcactcgcacgtcgcacccgctactctcatatacaccgagggcaaataattagacatatttgacacacacatagacaacatgtctagtgtaaaaaagattaaattgctaacatgttgacatcaaattttgtccaccagattcatcatctacagatgagtacaatttcacttgttctgacactgttttaaaccagcttgggttattcgtgctgggctgggcacgagctactactccaatgcacgcgttgtgcagctgggctatgggtgccacttgtgcacaagctgccagcggcttttcgccgcggccagcccagccttgaattcctggagagcctgcctggaggcccgtgcccggctccctcccgctcgctctccccagttgcgctccagctcagcagctcaggcgtgcgccgccgccagcgccgcctctcccataccgggcagcgcgggccccacccgctcgctgccccgcacgccagtgaccccggacccgataggcgtgggtgcgtggcgtcctcgtccgccgtcgcctacagcttagcttctccctcccgccagctcggaggcgccacgcgccgggctccgtccgaaacggccggctcgccggttcggaaactgacggcggggtggtacacgtcagcccagggccccgcacgtcagacggagagataggcatcggcgggggtatataaaagcgcctcctctcgtcctcctcgggccggcagtggcctagcttgttccattcatccactcagccggtgcacaccgtggagtggacggcggaggagcgctctctcgatgatccggccagcgccgtgggtgggcgccgggcaccgcgggcggggcggcgaggcgggcgcctgcaccgagagcctcggctccgagagtggcgacgtcggctgcgacgccgccgagatcgaccagttcttcccgccggcgccggctgctggcggcccggacgacgccggcgcagagccctgcattccggacctggcggcggcggcggcggcagggaaaaggcggcgcgggggcttccccccgccgatgccgcgcgcgtcggggccgctgttcctgcgcgcggagcgccgcggcggccgcctcatcctcaccgaggtgcgcgccgacgagcgtgagcgccgcgttgtgttccgcgccgagcgcgacggcggacgcctccgcctgcgcttcgccaacgacggcgacggccccgaggcggcaggtggtggtggcggcgccggcggtggaggcggggagctttgtcaggtggccgccggccggaggggcgtgcaggtcggcgcggtcatgatgggggctatctgaaggagggccgcccgatcgagtgacaacgagacgccggccggcagcagattgctgtcgttgccgatggtcgagagagagagagtcgtgttcgtcagcgagagccattttcgttcgttggttcgtttgatttgttttccattctatttttcatctgcaatggcgatgatcttgcaaagctgcattggagggagagagggagacgatagcgttatggatagggattatttagccgccgtgcgcccgtgcgtgcgcgtgcttaaccattgccttgcctcgtgattaaaattctgccatttttgcacacatccttgcctgctgggtgctggctgctgctacagatggtagctagctagtacagttcctgtaaagttaacaaaagctggcgcggagctagccgcagccaacaaataatgctcggggcacaggcagttgcaccggtggtccgtcgtcgcccgacgacggtcccggtcccggcggcggggtgagaccatgtcgcgaaaaggaaaaaaggaaacagagaatcgggcagaggctcacatcagcgaccttttttctcttggactgcgatgccgtgctcttgcatcagcttgggtctcccaaacagacagaaccgttggtttccgcctttcaggggtggcggcgccatggcggcgacccgtcttcccttttgctggttgcctattatatgcatggcggccgcaacggattcgatagggatggactgcagtgaatcactcaagcaaacgggatgatatgacgacatacaatttaataaaaaaaatatataagagagttttatgaaaaattaaattgttttacgaggatgaaactcttgatagactattttctaaataaataaaatattgatatggcaaccttgaaaacaatgtgataaaatacctattaaggcccgtttggtttgattga

SEQ ID NO: 2 - DNA, 2368 bp, Zea mays
ctgaacctctttaaaattgtcagtgtgctagttccctattaggggatgacaattggtatgaaacttgtggtacacaggttagaaaccggtactcactaaacccgcacccacatctattaccccgtgacgagtacaatatatttaggcactcgcacgtcgcacccgctactctcatatacaccgagggcaaataattagacatatttgacacacacatagacaacatgtctagtgtaaaaaagattaaattgctaacatgttgacatcaaattttgtccaccagattcatcatctacagatgagtacaatttcacttgttctgacactgttttaaaccagcctgggttactcgtgctgggctgggcacgagctactactccaatgcacgcgttgtgcagctgggctatgggtgccacttgtgcacaagctgccagcggcttttcgccgcggccagcccagccttgaattcctggagagcctgcctggaggcccgtgcccggctccctcccgctcgctctccccagttgcgctccagctcagcagctcaggcgtgcgccgccgccagcgccgcctctcccataccgggccgcgcgggccccacccactcgctgccccgcacgccagtgaccccggacccgataggcgtgggtgcgtggcgtcctcgtccgccgtcgcctacagcttagcttctccctcccgccagctcggaggcgccacgcgccgggttccgtccgaaacggccggctcgccggttcggaaactgacggcggggtggtacacgtcagcacagggccccgcacgtcagacggagagataggcatcggcgggggtatataaaagcgcctcctctcgtcctcctcgggccagcagtggcctagcttgttccattcatccacccagccggtgcacaccgtggagtggacggcggaggagcgctctctcgatgatccggccagcgccgtgggtgggcggcgggcaccgcgggcggggcggcgaggcgggcgcctgcaccgagagcctcggttctgagagcggcgacgtcggctgcgacgccgccgagatcgaccagttcttcccgccgctgccggctgctggcggcccggacgacgccggcgcagagccctgcattccggccctggctgcgtccgcggcggcggcagggaaaaggcggcgcgggggcttccccccgccgatgccgcgcgcgtcggggccgctgttcctgcgcgcggagcgccgcggcggccgcctcatcctcaccgaggtgcgcgccgacgagcgtgagcgccgcgtcgtgttccgcgccgagcgcgacggcggacgcctccgcctgcgcttcgccaacgacggcgacggccccgaggcggcaggtggtggtggtggcggcggcgccggcggtggaggggggggagctttgtcaggtggccgccggccggaggggcgtgcaggtcggcgcggtcatgatgggggctatctgaaggagggccgcccgatcgagtgacaacgagacgccggccggcagcagattgctgtcgttgccgatggtcgaaatcgagagagagagtcgtgttcgtcagcgagagccattttcgttcgttggttcgtttgatttgttttccattctatttttcatctgcaatggcgatgatcttgcaaagctgcattggagggagagagggagacgatagcgttatggatagggattattaagccgccgtgccccgtgcgtgcgcgtgcttaaccattgccttgcctcgtgattaaaattctgccatttttgcacacattcttgcctgctgggtgctggctgctgctacagatggtagctagctagtacagttcctgtaaagttaacaaaagctggcgcggagctagccgcagccaacaaataatgctcggggcacaggcagttgcactggtggtccgtcgtcgcccgacgacggtcccagcggcggggctgggcgaccatatcgcgaaaaggaaaaaggaaacagagaatcgggcagaggctcacatcagcgaccttttttctctgggacggcgatggcgtgctcttgcatcagcttgggtctctcaaacagaaccgttggtttccgcctttcaggggtggcggcgccatggcggcgacccgtcttcccttttttgctgcttgcctattatatgcatggcggccgcaacggattcatagggatggccggcagtgaatcattcaagcaaacgggatgatatgacatacaatttaatgaaaacgatatacgagagaggtttatgaaaaattaaaattttacgaggatgaaactcttgatagactgttttctaaacaaatattaatatggcaactctgaaaacaatgtgataaactacctattaa

SEQ ID NO: 3 - DNA, 1754 bp, Zea mays
ctgaacctcttttgtcagtgtgctagttccctattggggatgacaatgagtatgaaacttgcggtacacgggttaaaaactggtactcgctaaacccgcacccacatctatcaccccgtgacgagtacaatatatttaggcactcgcacgtcgcacccgctactctcatatacacccgagggcgaataattagacatatttcacacacacatagacaacatgtctagtataaaaaagattaaattgctaacatgttgacatcaaattttgtccaccagattcatcatctacagatgagtacaatttcacttgttctgacactgttttaaaccagcttgggttactcgtgctgggctgggcacgagctactactccaatgcacgcgttgtgcagctgggctatgggtgccacttgtgcacaagctgccagcggcttttcgccgcggccagcccagccttgaattcctggagagcctgcctggaggcccgtgcccggctccctcccgctcgctctccccggttgcgctccagctcagcagctcaggcgtgcgccgccgccagcgccgcctctcccataccgggccgcgcgggccccacccgctcgctgccccgcacgccagtgaccccggacccgataggcgtgggtgcgtggcgtcctcgtccgccgtcgcctacagcttagcttctccctcccgccagctcggaggcgccacgcgccgggctccgtccgaaacggccggctcgccggttcggaaactgacggcggggtggtacacgtcagcccagggccccgcacgtcagacggagagataggcatcggcgggggtatataaaagcgcctcctctcgtcctcctcgggccagcagtggcctagcttgttccattcatccacccacccagtcgttgccgatggtcgagagagaacgagagagagtgtgtcgtgttcgtcagcgagagccattttcgttcgttggttcgtttgatttgttttccattctatttttcatctgcaatggcgatgatcttgcaaagctgcattggagggagagagggagacgatagcgttatggatagggattattaagccgccgtgcgcccgtgcgtgcgcgtgcttaaccattgccttgcctcgtgattaaaattctgccatttttgcacacattcttgcctgctgggtgctgctacagatggtagctagctagtacagttcctgtaaagttaacaaaagctggcgcggagctagccgcagccaacaaataatgctcggggcacaggcagttgcaccggtggtccgtcgtcgcccgacgacggtcccggtcccggcggcggggtgagaccatgtcgcgaaaaggaaaaaaggaaacagagaatcgggcagaggctcacatcagcgaccttttttctcttggactgcgatgccgtgctcttgcatcagcttgggtctcccaaacagacagaaccgttggtttccgcctttcaggggtggcggcgccatggcggcgacccgtcttcccttttgctggttgcctattatatgcatggcggccgcaacggattcgatagggatggactgcagtgaatcactcaagcaaacgggatgatatgacgacatacaatttaataaaaaaatatataagagagttttatgaaaaattaaattgttttacgaggatgaaactcttgatagactattttctaaataaataaaatattgatatggcaaccttgaaaacaatgtgataaaatacctattaa

SEQ ID NO: 4 - protein, 173 aa, Zea mays
MIRPAPWVGAGHRGRGGEAGACTESLGSESGDVGCDAAEIDQFFPPAPAAGGPDDAGAEPCIPDLAAAAAAGKRRRGGFPPPMPRASGPLFLRAERRGGRLILTEVRADERERRVVFRAERDGGRLRLRFANDGDGPEAAGGGGGAGGGGGELCQVAAGRRGVQVGAVMMGAI

SEQ ID NO: 5 - protein, 184 aa, Zea mays
MIRPAPWVGGGHRGRGGEAGACTESLGSESGDVGCDAAEIDQFFPPLPAAGGPDDAGAEPCIPALAASAAAAGKRRRGGFPPPMPRASGPLFLRAERRGGRLILTEVRADERERRVVFRAERDGGRLRLRFANDGDGPEAAGGGGGGGAGGGGGGALSGGRRPEGRAGRRGHDGGYLKEGRPIE

SEQ ID NO: 6 - DNA, 21 bp, Artificial sequence
  Primer
cgcctacagcttagcttctcc

SEQ ID NO: 7 - DNA, 20 bp, Artificial sequence
  Primer
atcatcgagagagcgctcct

SEQ ID NO: 8 - DNA, 20 bp, Artificial sequence
  Primer
tctccctctctccctccaat

SEQ ID NO: 9 - DNA, 519 bp, Zea mays
atgatccggccagcgccgtgggtgggcgccgggcaccgcgggcggggcggcgaggcgggcgcctgcaccgagagcctcggctccgagagtggcgacgtcggctgcgacgccgccgagatcgaccagttcttcccgccggcgccggctgctggcggcccggacgacgccggcgcagagccctgcattccggacctggcggcggcggcggcggcagggaaaaggcggcgcgggggcttccccccgccgatgccgcgcgcgtcggggccgctgttcctgcgcgcggagcgccgcggcggccgcctcatcctcaccgaggtgcgcgccgacgagcgtgagcgccgcgttgtgttccgcgccgagcgcgacggcggacgcctccgcctgcgcttcgccaacgacggcgacggccccgaggcggcaggtggtggtggcggcgccggcggtggaggcggggagctttgtcaggtggccgccggccggaggggcgtgcaggtcggcgcggtcatgatgggggctatc

SEQ ID NO: 10 - DNA, 552 bp, Zea mays
atgatccggccagcgccgtgggtgggcggcgggcaccgcgggcggggcggcgaggcgggcgcctgcaccgagagcctcggttctgagagcggcgacgtcggctgcgacgccgccgagatcgaccagttcttcccgccgctgccggctgctggcggcccggacgacgccggcgcagagccctgcattccggccctggctgcgtccgcggcggcggcagggaaaaggcggcgcgggggcttccccccgccgatgccgcgcgcgtcggggccgctgttcctgcgcgcggagcgccgcggcggccgcctcatcctcaccgaggtgcgcgccgacgagcgtgagcgccgcgtcgtgttccgcgccgagcgcgacggcggacgcctccgcctgcgcttcgccaacgacggcgacggccccgaggcggcaggtggtggtggtggcggcggcgccggcggtggaggggggggagctttgtcaggtggccgccggccggaggggcgtgcaggtcggcgcggtcatgatgggggctatctgaaggagggccgcccgatcgag

SEQ ID NO: 11 - protein, 17 aa, Zea mays
RRVVFRAERDGGRLRLR